(19) United States
(12) Patent Application Publication
(10) Pub. No.: US 2022/0273460 A1
(43) Pub. Date: Sep. 1, 2022

(54) METHODS FOR PERFORMING AN ARTHROPLASTY OF THE SUBTALAR JOINT

(71) Applicant: Paragon 28, Inc., Englewood, CO (US)

(72) Inventors: Andrew Julian GOLDBERG, Radlett (GB); Kevin Alan DODD, Round Rock, TX (US); Mathew Richard ANDERLE, Greenwood Village, CO (US); Michael Edward CARROLL, Memphis, TN (US); Richard Matthew OBERT, Poway, CA (US); Gregory Charles MARIK, Germantown, TN (US)

(73) Assignee: Paragon 28, Inc., Englewood, CO (US)

(21) Appl. No.: 17/653,029

(22) Filed: Mar. 1, 2022

Related U.S. Application Data
(60) Provisional application No. 63/155,100, filed on Mar. 1, 2021.

(51) Int. Cl. A61F 2/46

(52) U.S. Cl. CPC A61F 2/4606; A61F 2/4684; A61F 2002/4207

(57) ABSTRACT

A method for performing an arthroplasty of a subtalar joint includes making at least one incision adjacent the subtalar joint, accessing the subtalar joint defined superiorly by a first articular surface and inferiorly by a second articular surface, and preparing at least one of the first articular surface and the second articular surface to form first and second interfacing surfaces. The method further includes identifying at least one landmark of the first interfacing surface and the second interfacing surface, determining at least one significant point on at least one of the first interfacing surface and the second interfacing surface relative to the at least one landmark, and placing at least one trial implant. The method also includes securing an implant positioned substantially inferior relative to the first interfacing surface and substantially superior relative to the second interfacing surface, stabilizing the subtalar joint, and closing the at least one incision.

### CROSS REFERENCE TO RELATED APPLICATIONS

This application claims priority benefit of U.S. Provisional Patent Application No. 63/155,100, filed Mar. 1, 2021, and entitled Methods For Performing An Arthroplasty Of The Subtalar Joint.

### TECHNICAL FIELD

The present disclosure relates to methods, devices, and instruments associated with performing arthroplasty procedures. The present disclosure relates to podiatric and orthopedic implants and surgery related to arthroplasty, arthrodesis, and/or arthroeresis of joints in the foot/ankle and/or procedures incorporating surrounding bones/soft tissue. More specifically, but not exclusively, the present disclosure relates to methods, implants, devices, and instruments relating to performing arthroplasty of the subtalar joint.

### BACKGROUND OF THE INVENTION

Many currently available methods, implants, instrumentation, devices, and systems for addressing joint trauma (acute and chronic, e.g., defect, gradual deterioration, etc.) do not completely address the needs of patients. Additionally, many currently available methods, implants, instrumentation, devices, and systems, for addressing joint trauma fail to account for properties of joint anatomy and associated mechanical and kinematic movement patterns/capabilities.

### SUMMARY

The present disclosure is directed toward implants, devices, and methods for use in maintaining, correcting and/or resurfacing joint surfaces.

A first aspect of the present disclosure is a method for performing arthroplasty of a subtalar joint. The method includes making at least one incision adjacent the subtalar joint, and accessing at least a portion of the subtalar joint, wherein the subtalar joint comprises a joint space defined superiorly by a first articular surface and inferiorly by a second articular surface. The method also includes preparing at least one of the first articular surface and the second articular surface to form a first interfacing surface and a second interfacing surface, identifying at least one landmark of the first interfacing surface and the second interfacing surface; and determining at least one significant point on at least one of the first interfacing surface and the second interfacing surface relative to the at least one landmark. The method also includes analyzing the joint space by placing at least one trial implant at least partially within the joint space, securing an implant such that the implant is positioned substantially inferior relative to the first interfacing surface and substantially superior relative to the second interfacing surface, stabilizing the subtalar joint, and closing the at least one incision.

According to the first aspect of the present disclosure, the method also includes positioning the trial implant such that at least a portion of the trial implant contacts the first and second interfacing surfaces.

According to the first aspect of the present disclosure, the method also includes analyzing the fit of the trial implant within at least a portion of the joint space.

According to the first aspect of the present disclosure, the method also includes placing a second implant trial at least partially within the joint space, wherein the implant trial comprised a first size and the second implant trial comprises a second size, wherein the second size is different from the first size.

According to the first aspect of the present disclosure, the method also includes analyzing a first position and a first function of the implant after the implant has been placed within at least a portion of the joint space.

According to the first aspect of the present disclosure, the method also includes confirming the first position and function of the implant.

According to the first aspect of the present disclosure, the method also includes adjusting the implant within at least a portion of the joint space such that the implant assumes a second position and a second function, wherein the second position and function are different than the first position and function.

According to the first aspect of the present disclosure, the method also includes stabilizing the subtalar joint by manipulating one or more soft tissue structures adjacent the subtalar implant after the implant has been placed and fixated at least partially within the joint space to reach a first stability.

According to the first aspect of the present disclosure, the method also includes confirming stability of the subtalar joint.

According to the first aspect of the present disclosure, the method also includes securing the implant by placing at least a first implant component adjacent the first interfacing surface and placing a second implant component adjacent the second interfacing surface.

According to the first aspect of the present disclosure, the method also includes placing a third implant component at least partially within the joint space such that the third implant component is positioned substantially inferior relative to the first implant component and substantially superior relative to the second implant component.

According to the first aspect of the present disclosure, the method also includes the at least one significant point being least one center of rotation disposed on the second interfacing surface.

According to the first aspect of the present disclosure, the method also includes aligning one or more components of the subtalar joint.

According to the first aspect of the present disclosure, the method also includes aligning the subtalar joint by manipulating at least one of a talus and a calcaneum such that at least a head of the talus is disposed substantially superior relative to at least a portion of the calcaneum.

According to the first aspect of the present disclosure, the method also includes the at least one landmark being one or more facets substantially disposed on a superior surface of the calcaneum.

According to the first aspect of the present disclosure, the method also includes the at least one landmark being one or more facets substantially disposed on a superior surface of the calcaneum.

According to the first aspect of the present disclosure, the method also includes the first interfacing surface being an inferior surface of the talus and the second interfacing surface being a superior surface of the calcaneum.

According to the first aspect of the present disclosure, the method also includes manipulating one or more soft tissue structures.

A second aspect of the present disclosure is a method for performing arthroplasty of a subtalar joint. The method includes accessing at least a portion of the subtalar joint, identifying at least one landmark of at least one of an inferior surface and a superior surface, and determining at least one center of rotation disposed on at least one of the inferior and superior surfaces. The method also includes placing at least one trial implant adjacent the inferior surface and the superior surface, and securing an implant such that the implant is positioned substantially between the inferior and superior surfaces.

A third aspect of the present disclosure is a method for performing arthroplasty of a subtalar joint including a first surface and a second surface. The method includes identifying at least one landmark of at least one of the first surface and the second surface, determining at least one significant point in relation to the at least one landmark, and securing an implant between the first and second surfaces in a first position, wherein the first position is based on a position of the at least one significant point.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a top view of a calcaneus shown in the transverse plane, in accordance with the present disclosure;

FIG. 2 is a bottom view of a talus shown in the transverse plane, in accordance with the present disclosure;

FIG. 3 is a flowchart of an aspect of a method for performing arthroplasty of a subtalar joint, in accordance with the present disclosure;

FIG. 4 is a flowchart of a first sub-method associated with the method of performing a subtalar arthroplasty shown in FIG. 4, in accordance with the present disclosure;

FIG. 5 is a flowchart of a second sub-method associated with the method of performing a subtalar arthroplasty shown in FIG. 4, in accordance with the present disclosure; and

FIG. 6 is a flowchart of a third sub-method associated with the method of performing a subtalar arthroplasty shown in FIG. 4, in accordance with the present disclosure.

### DETAILED DESCRIPTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in U.S. Pat. No. 10,117,749, issued on Nov. 6, 2018, European Patent No. 3756626 issued on Dec. 30, 2020, and European Patent Application No. 15770960.1A filed on Jul. 15, 2020; which are all hereby incorporated herein by reference in their entireties.

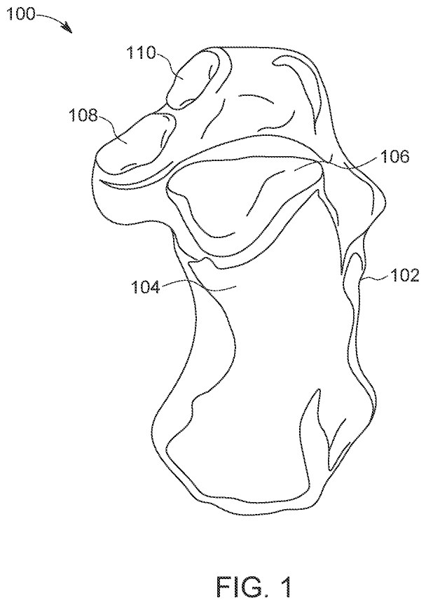

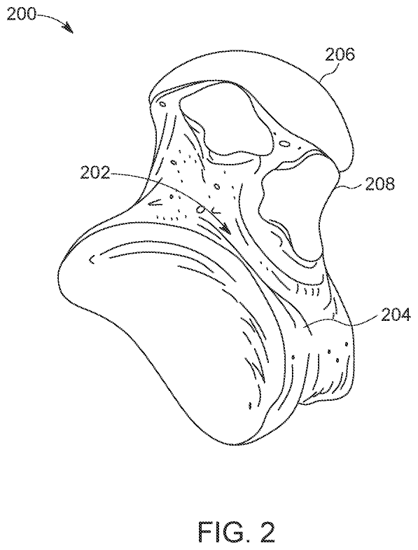

Referring to the drawings, wherein like reference numerals are used to indicate like or analogous components throughout the several views, and with particular reference to FIGS. 1-2, there is illustrated an exemplary embodiment of a right calcaneum (e.g., calcaneus) 100 shown from a superior view and a right talus 200 shown from an inferior view. The calcaneum 100 and the talus 200 are bones of the foot/ankle and two bones of the subtalar joint which is disposed substantially inferior (e.g., distal) relative to the ankle. According to some research and corresponding literature, the subtalar joint may be considered two joints based on one or more facets disposed on the calcaneum 100, which are shown with reference to FIG. 1 and described subsequently herein. These two joints include a first joint (the subtalar joint) including the posterior facet of the calcaneum 100, and a second joint (the talocalcaneonavicular joint) including the middle and/or anterior facet as well as the talonavicular joint (including the talus 200 and the navicular (not shown). However, for the purpose of this disclosure the subtalar joint is considered to include at least the calcaneum 100 (including the posterior, middle, and anterior facets), the talus 200, and the soft tissue disposed therebetween. The subtalar joint, which is positioned superior relative to the calcaneum 100 and inferior relative to the talus 200 is configured to permit/enable movement. Typically, the subtalar joint permits movement that includes at least inversion and eversion of the foot, where inversion is defined as a movement that causes the soles of the feet to face inwards (e.g., in a medial direction) and eversion is defined as a movement that causes the soles of the feet to face outwards (e.g., laterally). Further, inversion and eversion can include external and internal rotation, respectively, of the talus 200 relative to (e.g., on) the calcaneum 100. The calcaneum 100 and the talus 200 (and components thereof) are shown and described herein so as to provide context for various steps of the subsequently disclosed method of performing arthroplasty of the subtalar joint. Furthermore, it should be noted that the calcaneum 100 and the talus 200 as shown correspond to such bones found in the right foot of a human and that the structures shown and described herein may have different sizing and/or orientation with respect to the left foot of a human (but can be reasonably assumed to be equal and opposite) in the medial-lateral directions. It should be understood that the calcaneum 100 and the talus 200 may not be representative of the geometry of such bones of any/all humans/patients but are shown herein to be representative of the general geometry and features of the bones. For example, trauma, bone deformity, arthritis and other conditions can alter the geometry of such bones. As referred to herein, arthroplasty is defined as surgical reconstruction and/or joint replacement of a joint (in the context of this disclosure, the subtalar joint). It should be known that both the calcaneum 100 and the talus 200 may include other geometric features, interfaces with other portions of anatomy, and articulations in addition to those discussed herein.

The calcaneum 100 is shown to include a body 102 which includes a top surface 104. The top surface 104 of the calcaneum 100 may substantially include a superior surface of the calcaneum 100 and form at least a portion of an inferior portion and/or defining surface of the subtalar joint. In some aspects, the top surface 104 interfaces with cartilage positioned between the calcaneum 100 and the talus 200 (e.g., the subtalar joint) that may be removed in performing a subtalar joint arthroplasty. In some aspects, the top surface 104 may become one of two interfacing surfaces of a subtalar joint after arthroplasty is performed (e.g., a surface that interfaces with a subtalar joint implant). The top surface 104 may have various geometries depending on the patient and the condition of the calcaneum 100 (e.g., trauma, arthritis, deformity, etc.). As shown, the top surface 104 includes a posterior facet 106, a middle facet 108, and an anterior facet 110 as shown in FIG. 1. In some aspects, one or more of the posterior facet 106, the middle facet 108 and the anterior facet 110 may be less distinguishable than shown on the calcaneum 100 in FIG. 1. For example, in some aspects the middle facet 108 and the anterior facet 110 may be positioned closer to one another than shown in FIG. 1 such that the middle facet 108 and the anterior facet 110 overlap and/or abut one another (e.g., there is not a gap positioned anterior-lateral the middle facet 108). In some aspects, the calcaneum body 102, the posterior facet 206, the middle facet 108, the anterior facet 110 (as well as other components of the top surface 204 and the calcaneum 100) may be referenced as anthropometric markers (e.g., landscape markers, identifiers, etc.) in order to analyze movement and/or other kinematic properties of the calcaneum 100 and/or surrounding joints. It should also be understood that one or more events or conditions (e.g., trauma, arthritis, deformity, etc.) may alter the calcaneum 100 of a patient such that one or more of the features described has an altered geometry, is damaged, or is not present on the calcaneum 100 of a patient.

The talus 200 is shown to include a body 102 and a bottom surface 104. The top surface 204 of the talus 200 may substantially include an inferior surface of the talus 200 and form at least a portion of a superior portion and/or defining surface of the subtalar joint. In some aspects, the bottom surface 204 interfaces with cartilage positioned between the calcaneum 100 and the talus 200 (e.g., the subtalar joint) that may be removed in performing a subtalar joint arthroplasty. In performing arthroplasty of the subtalar joint, at least a portion of subcortical bone may be removed in order to place one or more components of an implant so as to promote maximum stability of the joint as well as other surrounding joints and structures. In some aspects, the bottom surface 204 may become one of two interfacing surfaces of a subtalar joint after arthroplasty is performed (e.g., a surface that interfaces with a subtalar joint implant). The bottom surface 204 may have various geometries depending on the patient and the condition of the calcaneum 100 (e.g., trauma, arthritis, deformity, etc.). The talus 200 is further shown to include a talar head 206 positioned at an anterior portion of the talus 200, with a talar neck 208 extending between the body 202 and the talar head 206. In some aspects, the talar body 202, the talar head 206, and the talar neck 208 (as well as other components of the bottom surface 204 and the talus 200) may be referenced as anthropometric markers (e.g., landscape markers, identifiers, etc.).) in order to analyze movement and/or other kinematic properties of the talus 200 and/or surrounding joints.

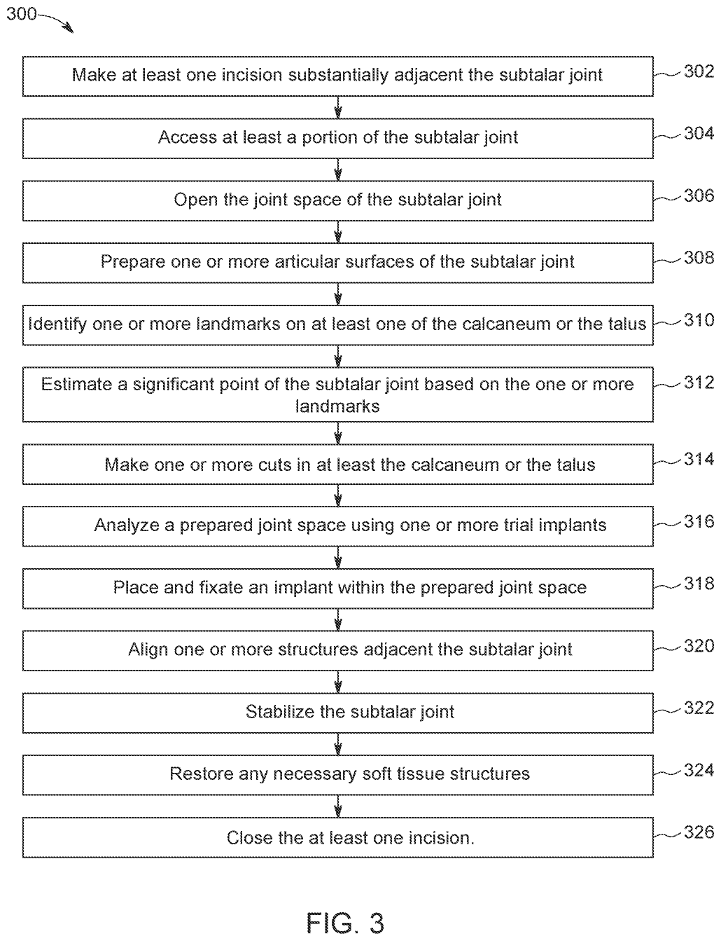

Referring now to FIGS. 3-6, there are illustrated flowcharts showing an exemplary embodiment for a process (e.g., method) of performing arthroplasty of the subtalar joint. The steps of the process shown in FIGS. 3-6 may reference one or more anatomical components, for example those shown and described with reference to FIGS. 1-2. However, it should be noted that the steps of the process shown and described with reference to FIGS. 3-6 is in no way limited to the geometric features of the anatomy shown in FIGS. 1-2 and may be applied to subtalar joints and components thereof having one or more alternate geometric features to those shown and described in FIGS. 1-2.

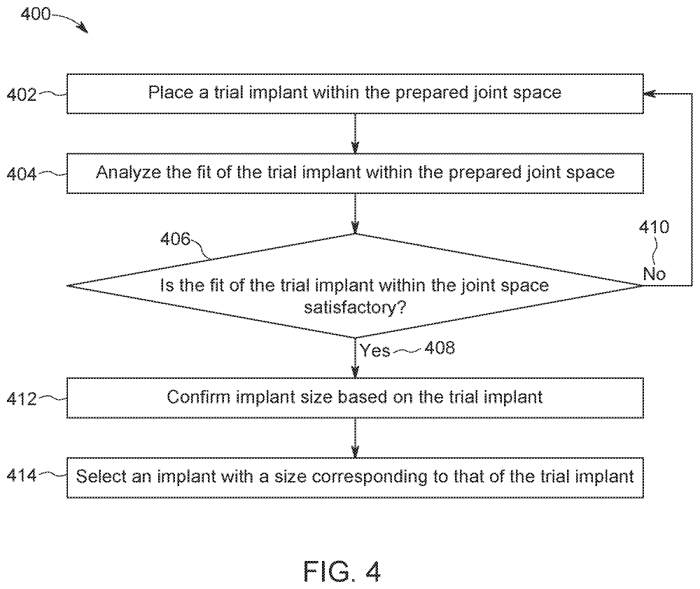

FIG. 3 shows a flowchart for a process 300 of performing subtalar arthroplasty, according to an exemplary embodiment. The process 300 is further shown to include three sub-processes shown and described with reference to FIGS. 4-6. Subtalar arthroplasty, as with all medical procedures, should be performed by one or more physicians, surgeons, or otherwise adequately trained healthcare providers and is shown and described as such. It should be understood that the steps of the process 300 (as well as the steps of the associated sub-processes) may be skipped, performed in an alternate order, repeated, or otherwise modified in performing a subtalar arthroplasty. In some aspects, alternate steps may be added and/or substituted for the steps shown and described herein with reference to the process 300. Furthermore, it should also be understood that the steps of the process 300 may not be limiting; that is to say that in some aspects additional steps may be performed in order to perform a subtalar arthroplasty. The steps of process 300 may be performed with one or more of the implants, instruments, devices, or other concepts discussed in the two issued patents and one pending patent application discussed previously and incorporated by reference herein in their entireties. Further, in some aspects one or more implants and/or pieces thereof may be implanted according to the process 300, for example an implant may include multiple pieces configured to interface with or adjacent to the posterior facet 106, the middle facet 108, the anterior facet 110, or other anatomical features. In some aspects, additional and/or alternative implants, instruments, devices, and/or other concepts to those incorporated by reference herein in their entireties may be implemented in conjunction with that that is shown and described herein.

The process 300 is shown to include a step 302 making at least one incision substantially adjacent a subtalar joint, according to the exemplary method shown and described in FIGS. 3-6. In some aspects, the at least one incision may be an incision extending from adjacent the distal tip of the fibula to the base of the fourth or fifth metatarsal such that the subtalar joint may be accessed laterally. However, in some aspects one or more circumstances (e.g., trauma, deformity, previous procedures, minimize surgical morbidity, etc.) may be present that are conducive to alternate incisions and/or directional approaches. For example, in some aspects the at least one incision may include an extended lateral incision/approach (e.g., in which a vertical limb meets a horizontal limb and the soft tissues on the side of the calcaneum are lifted off to access the lateral wall of the calcaneum and the subtalar joint). Additional incisions may also be made at, near, or adjacent the subtalar joint in order to provide optimal visualization of the subtalar joint and surrounding soft tissue structures. In some aspects, a subtalar arthroplasty may include removal of metalwork, for example after trauma where previous metalwork is in the calcaneum and/or talus and requires removal during a subtalar arthroplasty procedure, and accordingly the step 302 may include making one or more incisions configured to facilitate the removal of said metalwork. Further, in some aspects other alternate incisions and/or approaches may be taken, for example a posterior-lateral approach and/or a posterior approach (which, in some aspects, may require manipulating/taking down/severing the Achilles tendon). In some aspects, incisions may be made that are not directly adjacent the joint. Additionally, in some aspects a patient may have one or more conditions or circumstances that warrant a medial approach, for example trauma has occurred to one or more portions of the foot, ankle, and/or lower leg that prohibit approaches/incisions in directions other than the medial direction relative to the subtalar joint. Accordingly, in such circumstances a medial incision may be made. It should be understood that the step 302 may also include multiple incisions being made which may include a combination of two or more of the aforementioned incisions and/or alternate incisions (e.g., a posterior and a medial incision, a medial and a lateral incision, etc.).

The process 300 is shown to include a step 304 accessing at least a portion of the subtalar joint, according to the exemplary method shown and described in FIGS. 3-6. The step 304 may be performed directly after the step 302 as shown and described previously, for example at least a portion of the subtalar joint may be accessed via the at least one incision made in the step 302. Further, the step 304 may in some aspects be dependent on the one or more incisions made in the step 302 (e.g., if a lateral incision was made in the step 302, then the step 304 may include accessing the subtalar joint from a lateral direction). It should also be noted that the step 304 may include the manipulation, repositioning, and/or resection of one or more soft tissue structures disposed between the one or more incision made in the step 302. For example, if a lateral incision was made in the step 302, the step 304 may include manipulating, repositioning, bluntly dissecting, etc. one or more of the calcaneofibular ligament, the anterior talofibular ligament, and/or the extensor digitorium brevis (and/or other soft tissue structures). The soft tissue structures that may be contacted or otherwise addressed in the step 304 may be dependent on the one or more incisions made in the step 302, for example if a posterior approach is taken as opposed to a lateral the Achilles tendon may be contacted.

The process 300 is shown to include a step 306 opening a joint space of the subtalar joint, according to the exemplary method shown and described in FIGS. 3-6. The step 306 may include implementing one or more instruments common to arthroplasty procedures including, for example laminar spreaders, bone distractors over wires, elevators, osteotomes, sensors for ligament balancing, tracking arrays for bony alignment, PSI components, etc. Similar to the step 304, the step, 306 may be dependent on the one or more incisions made in the step 302. For example, the instruments and approach implemented in opening the joint space may be dependent on whether the joint is being accessed from a lateral, posterior lateral, posterior, medial, or other directional approach. Further, the step 306 and/or the instruments implemented in conjunction 306 may be dependent on the geometric features of the subtalar joint and components thereof (e.g., the calcaneum and the talus). For example, in accessing the joint laterally the physician may implement an instrument that is conducive to contact with the talus at or adjacent the sinus tarsi (disposed on the talus, but typically positioned adjacent the middle facet). Conversely, in accessing the subtalar joint posteriorly the physician may implement one or more instruments conducive to contacting the posterior facet as shown and described with reference to FIG. 1.

The process 300 is shown to include a step 308 preparing one or more surfaces of the subtalar joint, according to the exemplary method shown and described in FIGS. 3-6. As mentioned previously with reference to the steps 304 and 306, the step 308 may depend on the one or more incisions made in the step 302. For example, if the subtalar joint is accessed laterally, step 308 may include preparing the lateral-most portions of one or more surfaces (e.g., articular, interfacing, etc.) surfaces of the subtalar joint prior to preparing the medial-most portions. With reference to FIGS. 1-2, the top surface 104 and the bottom surface 204 of the calcaneum 100 and the talus 200, respectively, typically interface with cartilage and a subtalar arthroplasty procedure typically includes removing at least some (or in some aspects, all) of said cartilage and/or subchondral bone from the aforementioned surfaces. After the aforementioned cartilage is removed, the top surface 104 of the calcaneum 100 and the bottom surface 204 of the talus 200 (which may be resected and/or otherwise cut in subsequent steps) may contact the majority of a subtalar implant that is implanted and fixated between the calcaneum 100 and the talus 200 (and thus interfacing with the top surface 104 and the bottom surface 204). In some aspects, the step 308 may further include implementing one or more instruments in order to modify the top surface 104 of the calcaneum 100 and/or the bottom surface 204 of the talus 200. For example, in some aspects a physician may score (e.g., create an uneven texture by creating one or more depressions in the top surface 104 and/or the bottom surface 204) so as to promote fixation of and/or further preparation of the top surface 104 and/or the bottom surface 204 for arthroplasty.

The process 300 is shown to include a step 310 identifying one or more landmarks on at least one of the calcaneum or the talus, according to the exemplary method shown and described in FIGS. 3-6. The step 310 may include identifying one or more of the geometric features of the calcaneum 100 and/or the talus 200 shown and described with reference to FIGS. 1-2, for example the posterior facet 106, the middle facet 108, the anterior facet 110, the talar head 206, the talar neck 208, etc. In some aspects the step 310 may be dependent on one or more of the previous steps of the process 300 in that the landmarks that may be identifiable on the calcaneum 100 and/or the talus 200 may be dependent on the directional approach taken by a physician (e.g., dependent on the one or more incisions made and whether said incisions were made laterally, posterior-laterally, posterior, medial, etc. relative to the subtalar joint). In some aspects, the step 312 may further include the implementation of one or more imaging methods. For example, fluoroscope, CT, MRI, X-ray, or other imaging technologies may be implemented in order to capture images of the calcaneum 100, the talus 200 (and/or portions thereof), and/or anatomical components adjacent the subtalar joint for analysis by one or more physicians. As mentioned previously, in some aspects one or more components of the calcaneum 100, the talus 200, or other anatomical features adjacent the subtalar joint may not be present or may not be identifiable (e.g., trauma, degeneration, deformity, etc.). In some aspects, the step 310 may include identifying one or more of the aforementioned landmarks with respect to one or more of the other aforementioned landmarks (or other anatomical landmarks entirely). For example, if the middle facet 108 and the anterior facet 110 abut one another, the posterior facet 108 may be identified as a landmark and furthermore may be identified (e.g., measured, mapped, etc.) with respect to landmarks of the calcaneum 100 other than the middle facet 108 and the anterior facet 110 or with respect to landmarks of the talus 200, for example the talar head 206. The step 310 may also include the identification and measurement/estimation of one or more angles associated with the calcaneum 100, the talus 200, the subtalar joint, or other anatomical features/structures, for example the angle of Gissane. Soft tissue landmarks may also be identified including the soft tissue structures mentioned previously as well other soft tissue structures (which may depend on directional approach) for example the interosseous ligament, the deltoid ligament, peroneal tendons, calcaneofibular ligament, sural nerve, superficial peroneal nerve, neurovascular bundle (if medial approach) etc.

The process 300 is shown to include a step 312 estimating a significant point of the subtalar joint based on the one or more landmarks, according to the exemplary method shown and described in FIGS. 3-6. In some aspects, the step 312 may include estimating (or, in some aspects, determining) one or more significant points of the subtalar joint based on the one or more landmarks. The step 312 may further include the application of one or more algorithms, software programs, mathematical models, or other tools in order to analyze the landmarks identified (and in some cases imaged) in the step 310. Additionally, significant points of the subtalar joint may include and/or be referenced/identified with respect to one or more of the geometric features as shown and described previously with reference to FIGS. 1-2 (e.g., talar head, talar neck, one or more facets of the calcaneum, etc.). Further, other anatomical structures that may be positioned adjacent the subtalar joint (or in some research considered to be a part of the subtalar joint, for example the navicular) may also be incorporated in the identification of the one or more significant points. The significant points of the step 312 may include one or more centers of rotation, axis/axes, articulation points, pivot points (e.g., multiple pivot points along a line), one or more points along or central to a spline (e.g., piecewise polynomial function), ellipse, or other geometric curve or shape. Further, the one or more significant points of the step 312 may correspond to the aforementioned motion (e.g., inversion and eversion) of the subtalar joint. For example, the one or more significant points may correspond with one or more kinematic/mechanical properties and/or movements of the subtalar joint and, accordingly, correspond to one or more components of an implant to be implanted in a subtalar arthroplasty procedure. In some aspects, the one or more significant points may further correspond to one or more sizes of one or more components of an implant to be placed and fixated in the total arthroplasty procedure included in the process 300. For example, the location of one or more significant points (e.g., centers of rotation) estimated/determined/located on the top surface 104 of the calcaneum 100 may correspond to a size and/or placement/fixation location of one or more implant components. Similarly, one or more estimated/determined significant points and the location/proximity of said one or more significant points to one or more of the anatomic landmarks as shown and described previously may also correspond to sizing, placement, and/or selection of one or more components of an implant.

The process 300 is shown to include a step 314 making one or more cuts in at least the calcaneum or the talus, according to the exemplary method shown and described in FIGS. 3-6. The step 314, similar to one or more of the steps of the process 300 as described previously, may be dependent on the access direction (based on the one or more incisions of the step 302) of the subtalar joint. For example, the step 314 may include performing one or more cuts on the top surface 104 of the calcaneum and/or the bottom surface 204 of the talus 200. The approach, angle, instrumentation (e.g., saw, blade, drill, etc.) appropriate for such cuts may be dependent on the access and approach angle of the patient and the specific arthroplasty procedure. Depending on the approach angle and the access to the calcaneum 100 and the talus 200, different cuts and/or cutting patterns may need to be made/performed. In some aspects, a first cutting instrument may be implemented for a subtalar arthroplasty performed through a lateral incision, and a second instrument may be implemented for a different subtalar arthroplasty performed through a posterior incision. In some aspects, the one or more cuts of the step 314 may correspond to one or more components of an implant to be placed during a subtalar arthroplasty procedure. For example, a first cut or cuts may be made on the calcaneum 100 that correspond to a size, geometry, position, and function of a first implant component configured to be placed and/or fixated to/adjacent to a portion of the calcaneum 100, for example the top surface 104. Similarly, a second cut or cuts may be made to the talus 200 that correspond to a size, geometry, position, and function of a second implant component configured to be placed and/or fixated to/adjacent to a portion of the talus 200, for example the top surface 204 (or vice-versa, for example cuts are made to the talus 200 followed by cuts made to the calcaneum 100). In some aspects the step 314 may include the placement of one or more cut guides and/or jigs configured to guide one or more cuts.

The process 300 is shown to include a step 316 analyzing a prepared joint space using one or more trial implants, according to the exemplary method shown and described in FIGS. 3-6. The step 316 may be performed one or more times and, in some aspects may be an iterative process based on feedback received by the physician or other medical professional performing the step 316. Accordingly, the step 316 is shown as a sub-process 400 (referred to hereinafter as process 400) of analyzing a prepared joint space using one or more trial implants, which is shown and described with reference to FIG. 4. As with the step 316, the process 400 may also be repeated one or more times, in part or in full. As with the process 300, one or more steps of the process may be repeated, skipped, performed in an alternate order, or may be substituted for another step not shown in the exemplary embodiment of the process 400 of FIG. 4.

The process 400 is shown to include a step 402 placing a trial implant within the prepared joint space, according to an exemplary method shown and described in FIG. 4. The trial implant may be placed such that at least a first portion of the trial implant is in contact with the top surface 104 of the calcaneum and at least a second portion of the trial implant is in contact with the bottom surface 204 of the talus. It should be noted that in some aspects, trial implants may be placed both before and after cuts are made, surfaces are prepared, etc. In some aspects, the step 402 may include placing/securing/positioned one or more components of the trial implant relative to the one or more significant points identified in the step 312. For example, the step 402 may include placing a trial implant with features that correspond to one or more centers of rotation, pivot points, or other points included on an implant that correspond to a mechanism configured to mimic motion of a healthy subtalar joint. Accordingly, the step 402 may include placing/securing/positioning such a trial implant based on the one or more significant points identified in the step 312 The step 402 may also include the implementation of one or more instruments to facilitate placement and/or removal of the trial implant. In some aspects, one or more trial implants may be provided as components of a kit, system, or other set configured to include various components to be implemented in conjunction with the performance of a subtalar arthroplasty procedure. In some aspects, the trial implant may be one of multiple trial implants provided as a set, with each of the trial implants having a different size. The trial implant as referenced in the step 402 may also include a trial implant that consists of one or more components. The trial implant of the step 402 may be a component configured to have the same geometry and other spatial geometry as one or more components of an implant to be placed and fixated in a subtalar arthroplasty procedure. The step 402 may include placing the trial implant at least partially within the prepared joint space. That is to say that in some aspects one or more components of an implant may extend (e.g., laterally) beyond the dimensions/footprint of the calcaneum 100 and/or the talus 200. Accordingly, the implant trial may occupy a similar footprint, volume, and dimensions to those of one or more implants and/or components thereof (e.g., the implant of the disclosures referenced previously and incorporated herein in their entireties by reference). In some aspects, the step 402 may include assessing one or more possible corrected positions of one or more structures of the subtalar joint once the trial implant is placed.

The process 400 is shown to include a step 404 analyzing the fit of the trial implant within the prepared joint space, according to an exemplary method shown and described in FIG. 4. The step 404 may include the implementation of one or more imaging, placement/preoperateive planning technologies, or robotic/automated surgical technologies including but not limited to those described previously herein with reference to other steps of the process 300. The fit (e.g., position, orientation, sizing, etc.) of the trial implant may be analyzed by one or more physicians or medical professionals through various means. For example, the trial implant may have measurements taken with respect to surrounding anatomical features, for example soft tissue structures, the calcaneum 100, the talus 200, and features thereof (such as those shown and described previously herein). Accordingly, one or more instruments may be implemented in step 404 so as to facilitate the analysis and evaluation of the fit of the trial implant within the prepared joint space. In some aspects, such instruments may be provided as components of a kit such at that described previously herein. The step 404 may also include analysis and/or testing/balancing of soft tissue structures of the areas adjacent to the subtalar joint while the trial occupies at least a portion of the joint space.

The process 400 is shown to include a step 406 determining if the fit of the trial implant within the joint space is satisfactory, according to an exemplary method shown and described in FIG. 4. In some aspects, the step 406 may include various criteria (e.g., measurements, imaging data, etc.) that are incorporated in the determination of if the trial implant fit is satisfactory. Such a determination may also depend on the soft tissue analysis and balancing of the step 406, for example. The step 406 is followed by one of two possible steps. In some aspects, the step 406 results in a determination 408 that “yes” the fit of the trial implant within the joint space is satisfactory. In some aspects, the step 406 results in a determination 410 that “no” the fit of the trial implant is not satisfactory. The determination 410 results in a removal of the trial implant from the joint space and return to the first step of the process 400, step 402. Each time step 402 is repeated, a trial implant of a different size/volume/dimension (or at least a portion thereof) than that which was most recently removed from the joint space is placed within the joint space and the process 400 is repeated beginning with the step 402 until the determination 408 is made. In some aspects, a determination 410 of “no” may result to a return to the beginning of the process 300 and/or the step 302 for additional preparation.

The process 400 is shown to include a step 412 confirming implant size based on the trial implant, according to an exemplary method shown and described in FIG. 4. The step 412 directly follows the determination 408 that “yes”, the fit of the trial implant within the joint space is satisfactory. Upon such a determination, the step 412 is performed in order to identify a size of an implant and/or one or more components thereof that corresponds to that of the trial implant that provided the satisfactory fit within the joint space. In some aspects, measurements and/or imaging data that was applied to the decision of the step 406 may be further analyzed in order to determine an implant size that will provide an implant fit consistent with the satisfactory trial implant fit.

The process 400 is shown to include a step 414 selecting an implant with a size corresponding to that of the trial implant, according to an exemplary method shown and described in FIG. 4. Following the selection of an implant size in the step 414, an implant is selected based on the sizing determined in the step 414. In some aspects, this may include identifying an implant of a single size, while in other aspects this may include identifying multiple components (e.g., a calcaneal and a talar) of an implant that are available in one or more different sizes (which in some aspects may be compatible with various sizes of one another). Following the selection of the implant of the step 414, the process 400 has been completed. However, as mentioned previously the process 400 may include one or more steps that may be repeated and/or be performed out of order. That is to say that if an implant size was selected but a physician was not confident said implant size corresponded to the trial implant that resulted in the determination 408, one or more steps of the process 400 may be repeated.

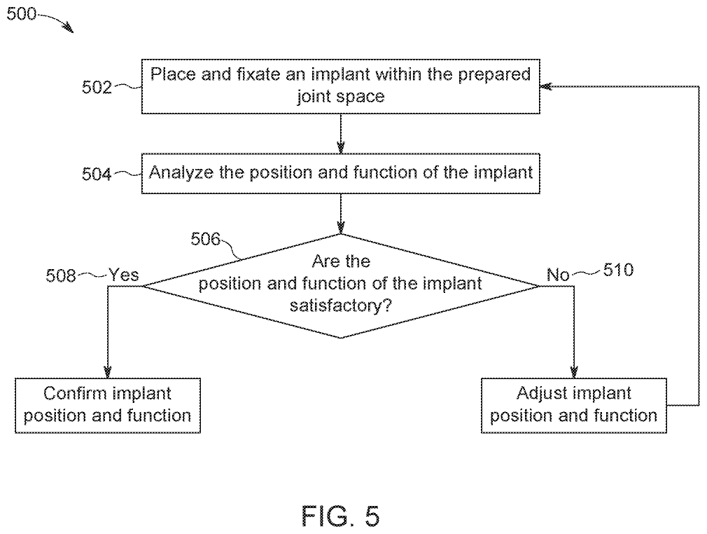

The process 300 is shown to include a step 318 placing and fixating (e.g., securing, using hardware, biologics, and/or other components, compositions, and/or techniques) an implant within the prepared joint space, according to the exemplary method shown and described in FIGS. 3-6. The step 318 may be performed one or more times and, in some aspects may be an iterative process based on feedback received by the physician or other medical professional performing the step 318. Accordingly, the step 318 is shown as a sub-process 500 (referred to hereinafter as process 500) of placing and fixating an implant withing the prepared joint space, which is shown and described with reference to FIG. 5. As with the step 318, the process 500 may also be repeated one or more times, in part or in full. As with the process 300, one or more steps of the process may be repeated, skipped, performed in an alternate order, or may be substituted for another step not shown in the exemplary embodiment of the process 500 of FIG. 5.

The process 500 is shown to include a step 502 placing and fixating an implant within the prepared joint space, according to an exemplary method shown and described in FIG. 5. In some aspects, the step 502 may include implementing one or more means to secure one or more implant components. For example, the step 502 may include implementing screws or other hardware configured to couple one or more components of the implant with the calcaneum 100 (e.g., such that the implant is adjacent/interfaces with the top surface 104) and/or the talus 200 (e.g., such that the implant is adjacent the bottom surface 204). In some aspects, a biologic solution such as a bone cement or similar coupling aid/adhesive agent may be implemented in conjunction with the aforementioned hardware. The step 502 may include securing one or more components to the aforementioned structures, for example a first component coupled/secured with the calcaneum 100 and a second component coupled/secured with the talus 200. In some aspects, the step 502 may include placing/securing/positioned one or more components of the implant relative to the one or more significant points identified in the step 312. For example, the step 502 may include placing an implant with one or more centers of rotation, pivot points, or other points that correspond to a mechanism configured to mimic motion of a healthy subtalar joint. In some aspects, one or more components of the implant may include a porous or other similar structure configured to promote bone ingrowth and/or ongrowth. Accordingly, the step 502 may include placing/securing/positioning such an implant based on the one or more significant points identified in the step 312. Further, in some aspects the step 502 may include securing a third implant component, for example an intermediate component disposed between the calcaneum 100 and the talus 200 and in contact/interfacing with a calcaneal implant component and a talar implant component. In some aspects, the step 502 may include the implementation of one or more instruments (some or all of which may be provided in a kit or system with the trial implants, the implant (s), and/or other components). For example, a first instrument may be implemented in securing a first implant component, while a second tool is subsequently implemented in securing a second implant component. In some aspects, the implant may be provided as a single component (e.g., already assembled or integral).

The process 500 is shown to include a step 504 analyzing the position and function of the implant, according to an exemplary method shown and described in FIG. 5. The step 504 may include the implementation of one or more imaging technologies the same as or similar to others described previously herein. For example, following implantation/fixation/securing of the implant with the calcaneum 100 and/or the talus 200, one or more physicians may analyze the position of the implant relative to various anatomical features (e.g., those shown and described previously, for example with reference to FIGS. 1-2). In some aspects, the step 504 may include data collection, data analysis, and anatomic measurements incorporating the implant. Further, the step 504 may also include the manipulation of the implant/joint (in some aspects, while using imaging technology) through normal subtalar range of motion (e.g., inversion and eversion) to evaluate if the implant meets various parameters, for example range of motion to a specified degree range (which may also include the implementation of one or more robotic/automated surgical technologies).

The process 500 is shown to include a step 506 determining if the position and function of the implant are satisfactory, according to an exemplary method shown and described in FIG. 5. The step 506 may be dependent on data, manipulation, and other outcomes of the step 504. For example, if the implant is measured to be in the correct place relative to anatomic features/landmarks and/or the one or more estimated/determined significant points, a physician may deem that the position and function of the implant are satisfactory. A physician may also account for range of motion when manipulating the joint and/or implant. However, if the implant is measured to be incorrectly placed (for example, does not meet the criteria mentioned above), a physician may elect to perform further testing or collect additional data, for example.

The process 500 is shown to include a determination 508 of “yes” the position and function of the implant are satisfactory, or a determination 510 of “no” the position and/or the function of the implant is/are not satisfactory, according to an exemplary method shown and described in FIG. 5. In the event that the determination 508 is made and “yes”, the position and function of the implant are determined to be satisfactory, the process 500 is complete with the implant position and function confirmed. However, in the event that the determination 510 is made and “no”, the position and/or function of the implant is/are not determined to be satisfactory, the position and function of the implant may be adjusted and/or step 502 may be repeated. In some aspects, this may include repositioning of the implant and/or other hardware. The process 500 may then be repeated until the step 506 is completed and the determination 508 is reached. However, as mentioned previously the process 500 may include one or more steps that may be repeated and/or be performed out of order. That is to say that if the determination 508 was made and a physician subsequently questions the position and/or function of the implant, one or more steps of the process 500 may be repeated. In some aspects, a determination 510 of “no” may result to a return to the beginning of the process 300 and/or the step 302 for additional preparation.

The process 300 is shown to include a step 322 aligning one or more structures adjacent the subtalar joint, according to the exemplary method shown and described in FIGS. 3-6. The step 322 may include manipulation of the calcaneum 100, the talus 200, and/or other structures surrounding the subtalar joint (e.g., soft tissue) so as to achieve a desired alignment. In some aspects, a desired alignment may include aligning one or more portions of the calcaneum 100 inferior relative to one or more portions of the talus 200. For example, in order to correct and/or prevent flatfoot deformity, a physician may manipulate the calcaneum 100 and/or the talus 200 such that at least a portion of the calcaneum 100 is disposed inferior (e.g., directly inferior) the talar head 206 so as to prevent or minimize the risk of the talus 200 (e.g., talar head 206) “falling off” (e.g., moving in an inferior direction) from a desired position superior relative to the calcaneum 100. In some aspects, the step 322 may include alternate alignment configurations to address various conditions/deformities of the foot and/or ankle. In some aspects, the step 322 may be performed prior to bone preparation, and may be assessed/analyzed with the one or more trial implants as described previously.

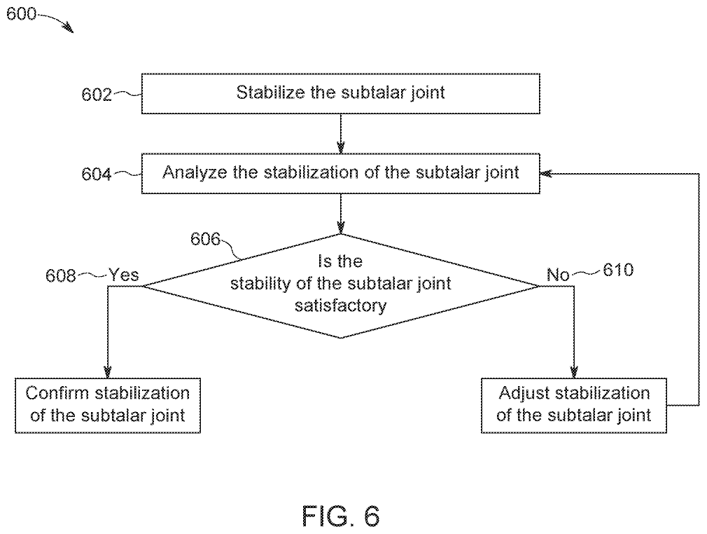

The process 300 is shown to include a step 322 stabilizing the subtalar joint, according to the exemplary method shown and described in FIGS. 3-6. The step 320 may be performed one or more times and, in some aspects may be an iterative process based on feedback received by the physician or other medical professional performing the step 320. Accordingly, the step 322 is shown as a sub-process 600 (referred to hereinafter as process 600) of stabilizing the subtalar joint, which is shown and described with reference to FIG. 6. As with the step 318, the process 600 may also be repeated one or more times, in part or in full. As with the process 600, one or more steps of the process may be repeated, skipped, performed in an alternate order, or may be substituted for another step not shown in the exemplary embodiment of the process 600 of FIG. 6.

The process 600 is shown to include a step 602 stabilizing the subtalar joint, according to the exemplary method shown and described in FIGS. 3-6. The step 602 may include incorporating one or more imaging technologies including but not limited to those shown and described previously in order to collect data directed to stability of the subtalar joint. In some aspects, the stability of the subtalar joint may be largely dependent on various positioning, tensioning, and balancing of forces present in various soft tissue structures adjacent the subtalar joint (e.g., ligaments, tendons, muscles, etc.). In some aspects, the step 602 may include implementing one or more instruments in order to manipulate one or more soft tissue structures and/or measure forces present in the aforementioned soft tissue structures. For example, one or more instruments may be provided in conjunction with the implant and other components with said one or more instruments configured to be implemented in order to manipulate (e.g., reposition, shift, etc.) soft tissue. In some aspects, the step 602 may also include balancing of soft tissues to address deformities, trauma, or degeneration present at or near the subtalar joint.

The process 600 is shown to include a step 604 analyzing the stabilization of the subtalar joint, according to the exemplary method shown and described in FIGS. 3-6. The step 604 may include the implementation of imaging technology the same as and/or similar to that mentioned with respect to the step 602 in order to analyze, measure, and collect data indicative of one or more forces or positions of the soft tissue structures adjacent the subtalar joint. In some aspects, instruments provided in a kit or system with the implant may be implemented in order to facilitate the analysis of the stability of the subtalar joint. Further, analyzing the stability of the subtalar joint may include manipulation of the subtalar joint (including the implant) so as to measure and observe behavior of soft tissue structures with respect to movement of the implant. In some aspects, the physician may have criteria (e.g., forces, positions, range of motion, etc.) which the various soft tissue structures must satisfy.

The process 600 is shown to include a step 606 determining if the stability of the subtalar joint is satisfactory, according to the exemplary method shown and described in FIGS. 3-6. The step 606 may be dependent on data, manipulation, and other outcomes of the step 604. For example, if the stability of the subtalar joint (e.g., soft tissue structures) is measured to meet criteria relative to anatomic features/landmarks and/or the one or more estimated/determined significant points, a physician may deem that the stability of the subtalar joint is satisfactory. A physician may also account for range of motion when manipulating the joint and surrounding soft tissue structures. However, if the subtalar joint is measured to be unstable and/or incorrectly balanced (for example, does not meet the criteria mentioned above), a physician may elect to perform further testing or collect additional data and/or repeat necessary steps to establish and confirm stability.

The process 600 is shown to include a determination 608 of “yes” the stabilization of the subtalar joint is satisfactory, or a determination 610 of “no” the stabilization of the subtalar joint is not satisfactory, according to an exemplary method shown and described in FIG. 6. In the event that the determination 608 is made and “yes”, the stabilization of the subtalar joint is determined to be satisfactory, the process 600 is complete with the stabilization of the subtalar joint confirmed. However, in the event that the determination 510 is made and “no”, the stabilization of the subtalar joint is not determined to be satisfactory, the stabilization of the subtalar joint be adjusted and/or step 502 may be repeated. In some aspects, this may include manipulation of one or more of the soft tissue structures relative to the subtalar joint/implant and/or the calcaneum 100 and/or the talus 200. The process 600 may then be repeated until the step 606 is completed and the determination 608 is reached. However, as mentioned previously the process 600 may include one or more steps that may be repeated and/or be performed out of order. That is to say that if the determination 608 was made and a physician subsequently questions the stability of the subtalar joint, one or more steps of the process 600 may be repeated (and/or steps 310-316 of the process 300). In some aspects, a determination 610 of “no” may result to a return to the beginning of the process 300 and/or the step 302 for additional preparation.

The process 300 is shown to include a step 324 restoring any necessary soft tissue structures, according to the exemplary method shown and described in FIGS. 3-6. The step 324, in some aspects, be dependent on the steps 302, 304, and 306. Depending on where the at least one incision was made and the soft tissue structures that were encountered/contacted in steps 304 and 306, similar and/or the same soft tissue structures may require restoration after the subtalar implant has been placed. For example, if the one or more incisions made included a lateral incision, a physician may restore one or more of the calcaneofibular ligament, the anterior talofibular ligament, and/or the extensor digitorium brevis. Similarly, if the one or more incisions made included a posterior incision, a physician may at least a portion of the Achilles and/or other tendons.

The process 300, as well as the three sub-processes included therein (and referred to as processes 400, 500, and 600) may also be performed/implemented in conjunction with a patient-specific implant (PSI) as well as other components of a PSI system (e.g., imaging technology, software, jigs, cut guides, instrumentation, etc.). For example, one or more sets of imaging data (e.g., collected using one or more of the imaging technologies mentioned herein) may be used to collect patient data prior to a subtalar arthroplasty procedure (e.g., the process 300). This data may then be used to create (e.g., 3-D print, machine, etc.) one or more PSI components such as those mentioned previously. For example, one or more components of a subtalar joint implant may include a geometry that is based on the patient data collected prior to the subtalar arthroplasty procedure. Similarly, the step 314 of making one or more cuts in at least the calcaneum or the talus may include the use of one or more PSI cut guides and/or jigs. Such cut guides/jigs may be created based on the patient data collected and configured to guide one or more cuts so as to access and/or avoid various anatomical features. It should also be noted that PSI instrumentation may also be implemented in one or more of the steps of the process 300.

The process 300 is shown to include a step 326 closing the at least one incision, according to the exemplary method shown and described in FIGS. 3-6. In some aspects, the step 326 may include closing one or more incisions using sutures or other fixation members and/or adhesives. Antibacterial agents may also be introduced at or near the incision in the step 326. The step 326 is dependent upon the at least one incision made in the step 302. In some aspects, only one incision was made in the step 302 and, thus, only one incision requires closing in the step 326. However, in other aspects multiple incisions may be made in the step 302 and accordingly multiple incisions require closing in the step 326.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. A method for performing an arthroplasty of a subtalar joint, the method comprising:
making at least one incision adjacent the subtalar joint;
accessing at least a portion of the subtalar joint, wherein the subtalar joint comprises a joint space defined superiorly by a first articular surface and inferiorly by a second articular surface;
preparing at least one of the first articular surface and the second articular surface to form a first interfacing surface and a second interfacing surface;
identifying at least one landmark on at least one of the first interfacing surface and the second interfacing surface;
determining at least one significant point on at least one of the first interfacing surface and the second interfacing surface relative to the at least one landmark;
placing at least one trial implant at least partially within the joint space;
securing an implant such that the implant is positioned substantially inferior relative to the first interfacing surface and substantially superior relative to the second interfacing surface;
stabilizing the subtalar joint; and
closing the at least one incision.

2. The method of claim 1, further comprising positioning the trial implant such that at least a portion of the trial implant contacts the first and second interfacing surfaces.

3. The method of claim 2, further comprising analyzing the fit of the trial implant within at least a portion of the joint space.

4. The method of claim 2, further comprising placing a second implant trial at least partially within the joint space, wherein the implant trial comprised a first size and the second implant trial comprises a second size, wherein the second size is different from the first size.

5. The method of claim 1, further comprising analyzing a first position and a first function of the implant after the implant has been placed within at least a portion of the joint space.

6. The method of claim 5, further comprising confirming the first position and function of the implant.

7. The method of claim 5, further comprising adjusting the implant within at least a portion of the joint space such that the implant comprises a second position and a second function, wherein the second position and function are different than the first position and function.

8. The method of claim 1, wherein stabilizing the subtalar joint comprises manipulating one or more soft tissue structures adjacent to the implant after the implant has been placed and fixated at least partially within the joint space to reach a first stability.

9. The method of claim 9, further comprising confirming stability of the subtalar joint.

11. The method of claim 1, wherein securing the implant comprises placing at least a first implant component adjacent the first interfacing surface and placing a second implant component adjacent the second interfacing surface.

12. The method of claim 11, further comprising placing a third implant component at least partially within the joint space such that the third implant component is positioned substantially inferior relative to the first implant component and substantially superior relative to the second implant component.

13. The method of claim 1, wherein the at least one significant point is at least one center of rotation disposed on the second interfacing surface.

14. The method of claim 1, further comprising aligning one or more components of the subtalar joint.

15. The method of claim 14, wherein aligning the subtalar joint comprises manipulating at least one of a talus and a calcaneum such that at least a head of the talus is disposed substantially superior relative to at least a portion of the calcaneum.

16. The method of claim 1, wherein the at least one landmark comprises one or more facets substantially disposed on a superior surface of the calcaneum.

17. The method of claim 1, wherein the first interfacing surface is an inferior surface of the talus and the second interfacing surface is a superior surface of the calcaneum.

18. The method of claim 1, further comprising manipulating one or more soft tissue structures.

19. A method for performing arthroplasty of a subtalar joint, the method comprising:
accessing at least a portion of the subtalar joint;
identifying at least one landmark on at least one of an inferior surface and a superior surface;
determining at least one center of rotation disposed on at least one of the inferior and superior surfaces;
placing at least one trial implant adjacent the inferior and superior surfaces; and
securing an implant such that the implant is positioned substantially between the inferior and superior surfaces.

20. A method for performing arthroplasty of a subtalar joint, the subtalar joint comprising a first surface and a second surface, the method comprising:
at least one landmark of at least one of the first surface and the second articular surface;
determining at least one significant point in relation to the at least one landmark; and
securing a subtalar implant between the first and second surfaces in a first position, wherein the first position is based on a position of the at least one significant point.
